(19) United States
(12) Patent Application Publication
(10) Pub. No.: US 2026/0060654 A1
(43) Pub. Date: Mar. 5, 2026

(54) METHOD FOR CONTROLLING TRANSDUCER OF ULTRASOUND PROBE AND ULTRASOUND IMAGING SYSTEM

(71) Applicant: GE Precision Healthcare LLC, Waukesha, WI (US)

(72) Inventors: Xin Ge, Wuxi (CN); Xinkang Liu, Wuxi (CN); Gengjie Du, Wuxi (CN); Binglong Liu, Wuxi (CN); Zihao Qian, Wuxi (CN); Xingti Liu, Wuxi (CN)

(21) Appl. No.: 18/821,719

(22) Filed: Aug. 30, 2024

(30) Foreign Application Priority Data
Aug. 30, 2024 (CN) 202311117185.X

(51) Int. Cl. A61B 8/00; A61B 8/14

(52) U.S. Cl. CPC A61B 8/4483; A61B 8/14; A61B 8/467

(57) ABSTRACT

A method for controlling a transducer of an ultrasound probe, including: controlling a plurality of basic elements of the transducer to simultaneously transmit ultrasonic signals, wherein the plurality of basic elements are arranged at intervals; detecting an echo signal of the ultrasonic signal transmitted by each of the plurality of basic elements; and controlling an activation state of an imaging function of the transducer based on a detection result of the echo signals. Further provided in the present application are an ultrasound imaging system and a non-transitory computer-readable medium.

### CROSS-REFERENCE TO RELATED APPLICATION

This application claim priority to Chinese Patent Application No. 202311117185.X, which was file on Aug. 31, 2023 at the Chinese Patent Office. The entire contents of the above-listed application are incorporated by reference herein in their entirety.

### TECHNICAL FIELD

The present invention relates to the field of medical imaging and, in particular, to a method for controlling a transducer of an ultrasound probe, an ultrasound imaging system and a non-transitory computer-readable medium.

### BACKGROUND

In ultrasound imaging technology, a probe is typically used to transmit an ultrasonic signal to a site to be scanned and receive an ultrasonic echo signal. Further, the echo signal is processed, obtaining an ultrasound image of the site to be scanned. Different ultrasound transducers may be required for different sites to be scanned. For example, for scanning of superficial sites, a high-frequency ultrasound transducer is typically used to improve the image resolution. For sites to be scanned at deeper depths, such as internal organs, the high-frequency ultrasound transducer is no longer suitable due to its insufficient penetration capability, and a lower-frequency ultrasound transducer will then be required to improve the penetration capability.

In current conventional workflows, a user's selection of an ultrasound transducer typically needs to be made manually. For example, there is a need in an ultrasound imaging system to select, through human-machine interaction, an ultrasound transducer that will perform ultrasound imaging to be activated, and then perform ultrasound imaging with the activated ultrasound transducer. If the transducer needs to be replaced during scanning, then the selection and activation are performed again in the ultrasound imaging system. Although some solutions for automatically activating an ultrasound transducer have been proposed, the accuracy of automatic activation is unsatisfactory compared to that of manual activation. The user is faced with various complicated workflows such as applying a coupling agent, moving a probe over the skin surface, and removing the probe from the skin surface during an ultrasound scanning process, which reduces the accuracy of determination of the automatic activation and is likely to cause a problem of erroneous activation or non-activation.

### SUMMARY

The aforementioned defects, deficiencies, and problems are solved herein, and these problems and solutions will be understood through reading and understanding the following description.

Provided in some embodiments of the present application is a method for controlling a transducer of an ultrasound probe, comprising: controlling a plurality of basic elements of the transducer to simultaneously transmit ultrasonic signals, wherein the plurality of basic elements are arranged at intervals; detecting an echo signal of the ultrasonic signal transmitted by each of the plurality of basic elements; and controlling an activation state of an imaging function of the transducer based on a detection result of the echo signals.

Some embodiments of the present application provides an ultrasound imaging system, comprising: an ultrasound probe and a processor. The ultrasound probe comprises a transducer. The processor is configured to perform the following method: controlling a plurality of basic elements of the transducer to simultaneously transmit ultrasonic signals, wherein the plurality of basic elements are arranged at intervals; detecting an echo signal of the ultrasonic signal transmitted by each of the plurality of basic elements; and controlling an activation state of an imaging function of the transducer based on a detection result of the echo signals.

Some embodiments of the present application further provide a non-transitory computer-readable medium, the non-transitory computer-readable medium having a computer program stored therein, the computer program having at least one code segment, and the at least one code segment executable by a machine to cause the machine to execute the following steps of the method for controlling a transducer of an ultrasound probe: controlling a plurality of basic elements of the transducer to simultaneously transmit ultrasonic signals, wherein the plurality of basic elements are arranged at intervals; detecting an echo signal of the ultrasonic signal transmitted by each of the plurality of basic elements; and controlling an activation state of an imaging function of the transducer based on a detection result of the echo signals.

It should be understood that the brief description above is provided to introduce, in a simplified form, concepts that will be further described in the detailed description. The brief description above is not meant to identify key or essential features of the claimed subject matter. The scope is defined uniquely by the claims that follow the detailed description. Furthermore, the claimed subject matter is not limited to implementations that solve any deficiencies raised above or in any section of the present disclosure.

### BRIEF DESCRIPTION OF THE DRAWINGS

The present application will be better understood by reading the following description of non-limiting embodiments with reference to the accompanying drawings, where:

FIG. 1 is a schematic diagram of an ultrasound imaging system according to some embodiments of the present application;

FIG. 2 is a schematic diagram of a method for controlling a transducer of an ultrasound probe according to some embodiments of the present application;

FIG. 3 is a schematic diagram of a transducer including a plurality of basic elements according to some embodiments of the present application;

FIG. 4 is a schematic diagram of a method for controlling a transducer of an ultrasound probe according to some other embodiments of the present application; and

FIG. 5 shows a schematic diagram of a wireless ultrasound probe according to some embodiments of the present application.

### DETAILED DESCRIPTION

Specific embodiments of the present invention will be described below. It should be noted that in the specific description of the embodiments, it is impossible to describe all features of the actual embodiments of the present invention in detail, for the sake of brief description. It should be understood that in the actual implementation process of any embodiment, just as in the process of any one engineering project or design project, a variety of specific decisions are often made to achieve specific goals of the developer and to meet system-related or business-related constraints, which may also vary from one embodiment to another. Furthermore, it should also be understood that although efforts made in such development processes may be complex and tedious, for a person of ordinary skill in the art related to the content disclosed in the present invention, some design, manufacture, or production changes made on the basis of the technical content disclosed in the present disclosure are only conventional technical means, and should not be construed as the content of the present disclosure being insufficient.

Unless otherwise defined, the technical or scientific terms used in the claims and the description should be as they are usually understood by those possessing ordinary skill in the technical field to which they belong. “First”, “second”, and similar words used in the present invention and the claims do not denote any order, quantity, or importance, but are merely intended to distinguish between different constituents. The terms “one” or “a/an” and similar terms do not express a limitation of quantity, but rather that at least one is present. The terms “include” or “comprise” and similar words indicate that an element or object preceding the terms “include” or “comprise” encompasses elements or objects and equivalent elements thereof listed after the terms “include” or “comprise”, and do not exclude other elements or objects. The terms “connect” or “link” and similar words are not limited to physical or mechanical connections, and are not limited to direct or indirect connections.

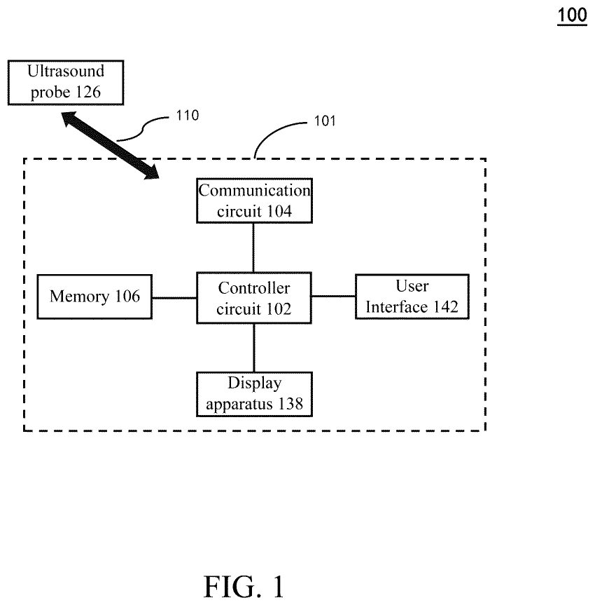

FIG. 1 shows a schematic block diagram of an embodiment of an ultrasound imaging system 100. The ultrasound imaging system 100 may include a host 101 and an ultrasound probe 126, where the ultrasound probe 126 is connected to the host 101 via a link 110. Further, the host 101 may include a controller circuit 102, a display apparatus 138, a user interface 142 and a memory 106 that are connected to a communication circuit 104.

The controller circuit 102 is configured to control operation of the ultrasound imaging system 100. The controller circuit 102 may comprise one or more processors. Optionally, the controller circuit 102 may comprise a central processing unit (CPU), one or more microprocessors, a graphics processing unit (GPU), or any other electronic component capable of processing inputted data according to a specific logic instruction. Optionally, the controller circuit 102 may comprise and/or represent one or more hardware circuits or circuit systems, and the hardware circuit or circuit system comprises, is connected to, or comprises and is connected to one or more processors, controllers, and/or other hardware logic-based apparatuses. Additionally or alternatively, the controller circuit 102 may execute an instruction stored on a tangible and non-transitory computer-readable medium (e.g., the memory 106).

The controller circuit 102 may be connected to and/or control the communication circuit 104. The communication circuit 104 is configured to receive and/or transmit information along a bidirectional communication link with one or more alternate ultrasound imaging systems, remote servers, etc. The remote server may represent patient information, a machine learning algorithm, a remotely stored medical image from a previous scan, and/or a diagnosis and treatment period of a patient, etc. The communication circuit 104 may represent hardware for transmitting and/or receiving data along a bidirectional communication link. The communication circuit 104 may include a transceiver, a receiver, etc., and an associated circuit system (e.g., an antenna) for communicating (e.g., transmitting and/or receiving) with the one or more alternate ultrasound imaging systems, remote servers, etc., by using a wired and/or wireless means. For example, protocol firmware for transmitting and/or receiving data along a bidirectional communication link may be stored in the memory 106 accessed by the controller circuit 102. The protocol firmware provides network protocol syntax to the controller circuit 102 so as to assemble a data packet, establish and/or segment data received along the bidirectional communication link, and so on.

The bidirectional communication link may be a wired (e.g., by means of a physical conductor) and/or wireless communication (e.g., utilizing a radio frequency (RF)) link for exchanging data (e.g., a data packet) between the one or more alternative ultrasound imaging systems, remote servers, etc. The bidirectional communication link may be based on a standard communication protocol, such as Ethernet, TCP/IP, Wi-Fi, 802.11, a customized communication protocol, Bluetooth, etc.

The controller circuit 102 is operatively connected to the display apparatus 138 and the user interface 142. The display apparatus 138 may include one or more liquid crystal display apparatuses (e.g., light emitting diode (LED) backlights), organic light emitting diode (OLED) display apparatuses, plasma display apparatuses, CRT display apparatuses, and the like. The display apparatus 138 may display patient information, one or more medical images and/or videos, a graphical user interface, or a component received by the display device 138 from the controller circuit 102, one or more 2D, 3D, or 4D ultrasound image data sets from ultrasound data stored in the memory 106, or an anatomical measurement, a diagnosis, processing information, and the like currently acquired in real time.

The user interface 142 controls the operation of the controller circuit 102 and the ultrasound imaging system 100. The user interface 142 is configured to receive an input from a clinician and/or an operator of the ultrasound imaging system 100. The user interface 142 may include a keyboard, a mouse, a trackball, a touch pad, one or more physical buttons, and the like. Optionally, the display apparatus 138 may be a touch screen display apparatus that includes at least a portion of the user interface 142. For example, a portion of the user interface 142 may correspond to a graphical user interface (GUI) that is generated by the controller circuit 102 and is shown on the display apparatus 138. The touch screen display apparatus may detect the presence of a touch from the operator on the display apparatus 138, and may also identify the location of the touch relative to the surface area of the display apparatus 138. For example, a user may select, by touching or contacting the display apparatus 138, one or more user interface components of the user interface (GUI) shown on the display device. User interface components may correspond to icons, text boxes, menu bars, etc., shown on the display apparatus 138. A clinician may select, control, and use a user interface assembly, interact with the same, and so on, so as to send an instruction to the controller circuit 102 to perform one or more operations described in the present application. For example, a touch may be applied using at least one among a hand, a glove, a stylus, and the like.

With continued reference to FIG. 1, the ultrasound imaging system 100 may include the ultrasound probe 126. The ultrasound probe 126 may include elements (not shown) such as a transducer, a transmitter, a transmit beam former and a detector/SAP electronics. The detector/SAP electronics may be used to control the switching of the transducer elements. The detector/SAP electronics may also be used to group the transducer elements into one or more sub-holes. Configurations of the ultrasound probe 126 will also be described exemplarily below, where the ultrasound probe 126 may be any type of probe, including a linear probe, a curved array probe, or a 1.25D array, 1.5D array, 1.75D array or 2D array probe. According to a preferred embodiment of the present application, the ultrasound probe 126 may be a probe for volumetric imaging. For example, the probe 126 may be an electronic 4D (E4D) probe. In addition, the probe 126 may also be a mechanical probe, for example, a mechanical 4D probe or a hybrid probe. The ultrasound probe 126 may be configured to acquire 4D ultrasound data that includes information about how volumes change over time and may be processed to obtain volumetric ultrasound images related to sites to be imaged. It can be understood that each volume may include a plurality of 2D images or slices, and accordingly, the controller circuit may select a required 2D image from the volumetric ultrasound images.

The ultrasound probe 126 may be configured to acquire ultrasound data or information from tissues to be imaged (e.g., organs such as breasts and the heart, and corresponding skin surfaces outside organs, etc.) of a patient. The ultrasound probe 126 is communicatively connected to the controller circuit by the transmitter. The transmitter transmits a signal to the transmit beam former on the basis of acquisition settings received by the controller circuit 102. The acquisition settings may define the amplitude, pulse width, frequency, gain setting, scanning angle, power, time gain compensation (TGC), resolution, and the like of ultrasonic pulses transmitted by the ultrasound transducer. The transducer transmits a pulsed ultrasonic signal into a patient (e.g., a body). The acquisition settings may be defined by a user operating the user interface 142. The signal transmitted by the transmitter, in turn, drives the transducer.

The transducer may include a plurality of basic elements arranged in an array. The basic elements include a piezoelectric material to achieve mutual conversion of electrical energy and mechanical vibration. The transducer uses the basic elements to transmit the pulsed ultrasonic signal to a body (e.g., a patient) or a volume that corresponds to an acquisition setting along one or more scanning planes. The ultrasonic signal may include, for example, one or more reference pulses, one or more push pulses (e.g., shear waves), and/or one or more pulsed wave Doppler pulses. At least a portion of the pulsed ultrasonic signal is backscattered from a tissue to be imaged (e.g., an organ, bone, heart, breast tissue, liver tissue, cardiac tissue, prostate tissue, newborn brain, embryo, abdomen, etc.) to produce an echo. Depending on depth or movement, the echo is delayed in time and/or frequency, and received by the transducer. The ultrasonic signal may be used for imaging, for producing and/or tracking a shear wave, for measuring changes in location or velocity within the anatomical structure and a compressive displacement difference (e.g., strain) of tissue, and/or for treatment and other applications. For example, the ultrasound probe 126 may deliver low-energy pulses, and deliver medium-energy and high-energy pulses to produce shear waves during imaging and tracking, and deliver high-energy pulses during treatment.

The transducer uses the basic elements to convert received echo signals into electrical signals that can be received by the receiver. The receiver may include one or more amplifiers, analog/digital converters (ADCs), and the like. The receiver may be configured to amplify the received echo signal after appropriate gain compensation, and convert these analog signals received from each transducer element into a digitized signal that is temporally uniformly sampled. The digitized signals representing the received echoes are temporarily stored in the memory 106. The digitized signals correspond to the backscattered waves received by each transducer element at different times. After being digitized, the signal may still retain the amplitude, frequency, and phase information of the backscattered wave.

Optionally, the controller circuit 102 may retrieve the digitized signals stored in the memory 106 for use in a beam former processor. For example, the controller circuit 102 may convert the digitized signal into a baseband signal or compress the digitized signal.

In some embodiments, the controller circuit 102 may further include a beam forming processor. The beam forming processor may include one or more processors. If desired, the beam forming processor may include a central processing unit (CPU), one or more microprocessors, or any other electronic component capable of processing the input data according to specific logic instructions. Additionally or alternatively, the beam forming processor may execute instructions stored on a tangible and non-transitory computer-readable medium (e.g., the memory 106) to perform beam forming computation using any suitable beam forming method, such as adaptive beam forming, synthetic emission focusing, aberration correction, synthetic aperture, clutter suppression, and/or adaptive noise control, etc.

In some embodiments, the controller circuit 102 may further include a radio frequency (RF) processor. The beam forming processor executes beam forming on the digitized signals of the transducer elements, and outputs an RF signal. The RF signal is then provided to the RF processor for processing the RF signal. The RF processor may include one or more processors. If desired, the RF processor may include a central processing unit (CPU), one or more microprocessors, or any other electronic component capable of processing the inputted data according to specific logic instructions. Additionally or alternatively, the RF processor may execute instructions stored on a tangible and non-transitory computer-readable medium (e.g., the memory 106). Optionally, the RF processor may be integrated with and/or be part of the controller circuit 102. For example, operations described as being executed by the RF processor may be configured to be executed by the controller circuit 102.

The RF processor may generate, for a plurality of scanning planes or different scanning modes, different ultrasound image data types and/or modes, e.g., B-mode, color Doppler (e.g., color blood flow, velocity/power/variance), tissue Doppler (velocity), and Doppler energy, on the basis of a predetermined setting of a first model. For example, the RF processor may generate tissue Doppler data for multiple scanning planes. The RF processor acquires the information (e.g., I/Q, B-mode, color Doppler, tissue Doppler, and Doppler energy information) related to a plurality of data pieces, and stores the data information in the memory 106, where the data information may include time stamp and orientation/rotation information.

Optionally, the RF processor may include a composite demodulator (not shown) for demodulating the RF signal to generate an IQ data pair representing an echo signal. The RF or IQ signal data may be provided directly to the memory 106 so as to be stored (e.g., stored temporarily). If desired, an output of the beam forming processor may be delivered directly to the controller circuit 102.

The controller circuit 102 may be configured to process the acquired ultrasonic data (e.g., RF signal data or an IQ data pair), and prepare and/or generate an ultrasound image data frame representing an anatomical structure of interest so as to display the same on the display apparatus 138. The acquired ultrasonic data may be processed by the controller circuit 102 in real time when an echo signal is received in a scanning or treatment process of ultrasound examination. Additionally or alternatively, the ultrasonic data may be temporarily stored in the memory 106 in a scanning process, and processed in a less real-time manner in live or off-line operations.

The memory 106 may be used to store processed frames of acquired ultrasound data that are not scheduled to be immediately displayed, or may be used to store post-processed images (e.g., shear wave images and strain images), firmware or software corresponding to, for example, a graphical user interface, one or more default image display settings, programmed instructions, and the like. The memory 106 may store a medical image, such as a 3D ultrasound image data set of ultrasonic data, where such a 3D ultrasound image data set is accessed to present 2D and 3D images. For example, the 3D ultrasound image data set may be mapped to corresponding memory 106 and one or more reference planes. Processing of ultrasound data that includes the ultrasound image data set may be based in part on user input, e.g., a user selection received at the user interface 142.

The memory 106 includes a parameter, an algorithm, one or more protocols of ultrasound examination, data values, and the like used by the controller circuit 102 to execute one or more operations described in the present application. The memory 106 may be a tangible and non-transitory computer-readable medium such as a flash memory, a RAM, a ROM, an EEPROM, etc. The memory 106 may include a set of learning algorithms (e.g., a convolutional neural network algorithm, a deep learning algorithm, a decision tree learning algorithm, etc.) configured to define an image analysis algorithm. During execution of the image analysis algorithm, the controller circuit 102 is configured to identify a section (or a view or an anatomical plane) of an anatomical structure of interest in a medical image. Optionally, an image analysis algorithm may be received by means of the communication circuit 104 along one among bidirectional communication links, and stored in the memory 106. It can be understood that the anatomical structure of interest may be a specific anatomical feature in a site to be scanned, and may be, for example, a muscle, a blood vessel, a tissue to be subjected to intervention (e.g., a tumor), etc.

While the functions of the various components of the ultrasound imaging system 100 and the manner in which they cooperate with one another have been exemplarily described above, it will be appreciated that those skilled in the art, in light of the above teachings of the present disclosure, may also adjust the arrangement of the ultrasound imaging system 100. Specific implementations of the host 101, the ultrasound probe 126 and the link 110 of the ultrasound imaging system 100 are exemplarily described below.

In some embodiments, the ultrasound probe 126 may be a wired probe. Accordingly, the link 110 may be a cable connecting between the ultrasound probe 126 and the host 101. The ultrasound probe 126 includes a cable and a probe connector. The probe connector is configured to be detachably connected to a connector receptacle on the host 101. At this time, the cable as the link 110 can both provide a power input for the ultrasound probe 126 and be used for transmission of signals. Moreover, the ultrasound probe 126 may also be fixedly connected to the host 101 via the cable without the need for the probe connector.

In some other embodiments, the ultrasound probe 126 may be a wireless probe. The wireless probe may be built with a battery therein so as to provide electrical energy to the ultrasound probe 126 for ultrasound scanning. The wireless probe communicates with the host 101, e.g., the communication circuit 104, via any wireless connections in the art, such as WiFi or Bluetooth. In addition, some other functions may further be integrated within the wireless probe. For example, a beam former may be included to process the ultrasonic echo signals to some extent, so as to reduce the amount of data transmission and improving the effect of data transmission. Furthermore, a processor may further be integrated within the wireless probe for processing or even imaging of data, in which case the wireless probe as a whole may be regarded as an ultrasound imaging system.

In some embodiments, the host 101 may be a desktop ultrasound host. The desktop host may include a probe connector receptacle for physically connecting with the ultrasound probe 126. In general, a plurality of probe connector receptacles may be included, and accordingly, a plurality of ultrasound probes 126 to which the probe connector receptacles are simultaneously connected may also be included in practical use. Users may select appropriate ultrasound probes 126 according to practical scanning conditions when performing ultrasound scanning. In addition, the desktop host may also have a wireless transceiver module, such as a wireless transceiver module integrated on the communication circuit 104, for signal transmission with the wireless ultrasound probe 126. Furthermore, the host 101 may also be a portable ultrasound host. The portable ultrasound host is more lightweight and and convenient, thus providing ease of use.

In other embodiments, the host 101 may be a portable electronic device for use with a wireless probe. For example, the host 101 may be a non-dedicated ultrasound device such as a smart phone, a tablet computer. These non-dedicated ultrasound devices, in which an ultrasound imaging function is integrated, e.g., by installing ultrasound imaging-related applications, can be used for ultrasound imaging. In a process of performing ultrasound imaging, components such as a processor, a user interface, and a display of the non-dedicated ultrasound device itself can be invoked to perform the functions of the host 101 in the ultrasound imaging system 100.

The inventors have found that, when the user performs ultrasound scanning using ultrasound probes, the user typically needs to select an ultrasound probe required to be activated on the host before performing the ultrasound scanning. The operation of probe selection not only additionally increases workloads, but also for a novice unfamiliar with an ultrasound imaging system, there may also be errors in selecting an ultrasound probe that is actually required to be activated. In some prior art, solutions of automatically identifying an ultrasound probe or transducer required to be activated are proposed. However, in such automatic identification, an additional sensor may need to be introduced, thus increasing the complexity of the device. More importantly, the ultrasound probe is typically faced with complex operating environments, e.g., various tilt angles, motion attitudes, etc. The accuracy of the automatic identification will be greatly affected.

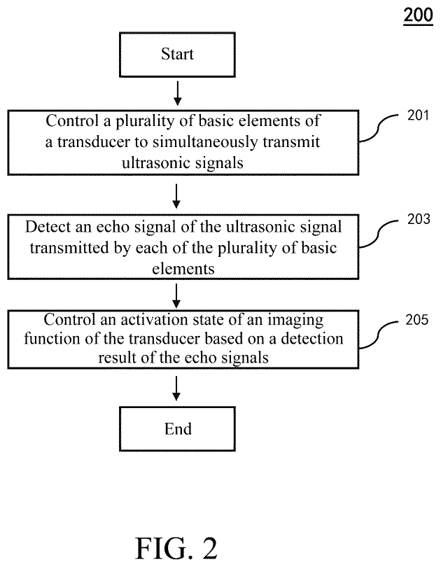

To address at least the technical problem described above, improvements are proposed in some embodiments of the present application. With reference to FIG. 2, a method 200 for controlling a transducer of an ultrasound probe according to some embodiments of the present application is shown, where the types and specific arrangements of the ultrasound probe and its transducer are as described in any embodiment herein.

In step 201, a plurality of basic elements of the transducer are controlled to simultaneously transmit ultrasonic signals, where the plurality of basic elements are arranged at intervals. This step may be executed by the processor. For example, it may be executed by the processor in the host 101 or the processor in the ultrasound probe 126 described in the embodiment above, where the transducer may include a basic-element array consisting of a number of basic elements. The plurality of basic elements may be selected from the basic-element array and belong to a part of the basic-element array. Moreover, the plurality of basic elements are arranged at intervals. Being arranged at intervals means that the plurality of basic elements are not contiguous and are spaced apart by other basic elements in between that are not used to control activation of an imaging function of the transducer. Such a configuration, on the one hand, can affect as little as possible the functions of other basic elements in the transducer, for example, an imaging function of a basic-element for imaging. On the other hand, it can also make the control of an activation state of an imaging function of the transducer in the present embodiment more accurate.

In step 203, an echo signal of the ultrasonic signal transmitted by each of the plurality of basic elements is detected. This process may also be controlled by the processor. Specifically, within a period of time after each of the plurality of basic elements transmits an ultrasonic signal, the processor uses the plurality of basic elements to receive with respect to whether the ultrasonic signal has an echo signal. It should be understood that there may be a number of cases in the receiving process described above. For example, each of the plurality of basic elements can receive the an signal, which case indicates that the plurality of basic elements achieve good acoustic coupling, and at the same time, since the plurality of basic elements are dispersedly distributed in the transducer, the above result will be more capable of reflecting the overall acoustic coupling of the entire transducer of the ultrasound probe. For another example, some of the plurality of basic elements can receive echo signals while some cannot receive echo signals, which case indicates that some of the plurality of basic elements have good acoustic coupling while some do not have good acoustic coupling. Alternatively, none of the basic elements receive echo signals, which indicates that the entire probe does not have good acoustic coupling.

In step 205, an activation state of an imaging function of the transducer is controlled based on a detection result of the echo signals. Specifically, the processor determines an acoustic coupling state of the transducer at that time according to a result of the echo signal received by each of the plurality of basic elements, and then determines whether the transducer at that time is in sufficient contact with a surface to be scanned and whether the user intends to perform ultrasound scanning with the transducer. If so, the imaging function of the transducer is controlled to be activated. If not, the imaging function of the transducer is controlled not to be activated.

Such a configuration can achieve rapid and accurate activation determination of the transducer in which the plurality of basic elements are located. Specifically, compared to external devices such as an angular velocity sensor, an acceleration sensor, and a gyroscope, according to the embodiments of the present application, a higher accuracy is provided, and no cost is additionally increased. The additional sensor not only increases the cost, but also is likely to cause erroneous determination when faced with complex scanning environments, e.g., operating conditions such as rapid movement and angle adjustment of the ultrasound probe hand-held by the user. In the solution of the present application, the plurality of basic elements used to assist in determining whether or not to activate the transducer are themselves part of the basic-element array of the ultrasound probe, and do not additionally increase the component costs. Moreover, the basic elements selected to assist with determination are distributed dispersedly and spaced apart from each other, and such determination can be more representative and less likely to cause erroneous determination. For example, in some cases, when a user inadvertently touches some of the basic elements of the transducer with fingers, the touched basic elements will be able to receive echo signals, and at this time, if it is determined whether the transducer is being used only by whether an echo signal is received or not, an erroneous conclusion will be drawn. In the embodiments of the present application, the echo signals of the plurality of basic elements distributed at intervals are used for comprehensive determination, which will be able to exclude the case of unexpected erroneous operations, so as to make the determination result more accurate.

How to control the activation state of the imaging function of the transducer based on the detection result of the echo signals is exemplarily described below.

In some embodiments, the imaging basic elements of the transducer can be controlled to transmit and receive the ultrasonic signals in response to the echo signals of the ultrasonic signals transmitted by two or more of the plurality of basic elements being detected, so as to activate the imaging function. The process may be implemented by the processor. Specifically, after detecting whether each of the plurality of basic elements receives the echo signal, the processor may determine as follows: the imaging function is activated when the signals transmitted by two or more of the plurality of basic elements are each capable of generating an echo signal. That the signals of two or more basic elements are each capable of having an echo signal indicates that the two or more basic elements achieve good acoustic coupling. Meanwhile, since the plurality of basic elements each are arranged at intervals, the good acoustic coupling of two or more basic elements can be representative to a greater extent as to whether or not the entire ultrasound probe is coupled to the surface of a site to be scanned, and such a solution can exclude a case that some of the basic elements generate acoustic coupling due to erroneous touching or the like. It should be understood that the imaging basic elements and the plurality of basic elements may similarly belong to the entire basic-element array of the transducer without the need for an additional structural design. Activation of the imaging function is any manner of imaging with an ultrasound probe in the art, which will not be repeated in the present application.

In some embodiments, the two or more basic elements include two basic elements that are farthest from each other in the plurality of basic elements. That is, although the processor may select all of the plurality of basic elements to assist with determination and activation control, in an alternative embodiment, it may also select some of the plurality of basic elements, for example, select at least two basic elements that are farthest from each other in the plurality of basic elements. Such an arrangement can ensure that some of the plurality of basic elements are selected in time for detection of the echo signals, and can also ensure the representativeness of the selection as much as possible. Determination of the acoustic coupling state of the two basic elements that are farthest from each other can reflect the acoustic coupling state of the entire transducer more accurately. In particular, even if two basic elements that are closer to each other (for example, same-side basic elements) have good acoustic coupling compared to two basic elements that are close to each other, it may be that only one side of the ultrasound probe is at that time in close proximity to the surface of the site to be scanned, and the overall fitting state may still not be reflected at that time.

In an alternative embodiment, the imaging basic-element of the transducer can also be controlled not to transmit and receive the ultrasonic signal in response to the echo signal of the ultrasonic signal transmitted by one or less of the plurality of basic elements being detected, so as not to activate the imaging function. Specifically, after detecting whether each of the plurality of basic elements receives the echo signal, the processor may determine as follows: not to activate the imaging function if the signal transmitted by only one or less of the plurality of basic elements can generate an echo signal. For example, when the ultrasonic signal of only one of the plurality of basic elements can generate an echo, it may be due to erroneous touching or a user's operation of applying an acoustic coupling agent or the like, which case does not represent the real intention of the user to activate the ultrasound probe. Alternatively, when the ultrasonic signals of none of the plurality of basic elements generate echoes, it indicates that the probe does not have any acoustic coupling, and the processor may control the imaging basic elements in the transducer not to transmit and receive ultrasonic signals. Such a configuration can make a determination quickly in an electrical energy saving manner as to whether to activate the imaging function. In particular, when ultrasound scanning is performed with a wireless probe, such a configuration can save electrical energy as much as possible for the wireless probe, so as to increase the endurance of the probe.

In some embodiments, the plurality of basic elements are different from the imaging basic elements. That is, the plurality of basic elements for assisting with activation of the imaging function and the imaging basic elements are different in the basic-element array. The plurality of basic elements for assisting with activation are not used for imaging, and the basic-element for imaging is not used for assisting with function activation. Such an arrangement, on the one hand, can ensure a stable ultrasound imaging quality, and on the other hand, can also select to make a real-time determination of acoustic coupling by using the plurality of basic elements simultaneously with ultrasound imaging by using the imaging basic elements, so as to adjust the activation state of the transducer in real time.

It should be understood that the different imaging function control manners described above may be arbitrarily selected, or may also be freely combined and used simultaneously.

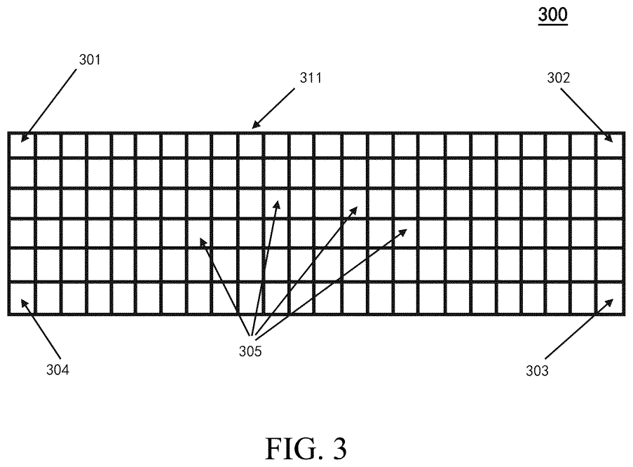

An arrangement of the plurality of basic elements for assisting in controlling whether to activate the imaging function of the ultrasound probe in the present application is exemplarily described below, With reference to FIG. 3, a schematic diagram of a transducer 300 including a plurality of basic elements according to some embodiments of the present application is shown.

As shown in FIG. 3, the transducer 300 includes a basic-element array, where the plurality of basic elements 301, 302, 303, 304 are configured to assist in controlling activation of an imaging function of the transducer 300 as described in any of the embodiments above. The specific control method is as described in any of the above embodiments, and will not be repeated here.

The positions of the plurality of basic elements 301-304 may be arranged in such a manner as shown in FIG. 3, that they are arranged at intervals at an edge of a contour 311 of the transducer 300. Such an arrangement can make the selection of the plurality of basic elements more representative, and can accurately reflect whether the entire transducer 300 is subjected to desired acoustic coupling and whether the user decides to use the transducer 300 to perform ultrasound scanning. On the other hand, the plurality of basic elements 301-304 for assisting with activation of the imaging function do not have a significant influence on the quality of an ultrasound image even if they are not used for the subsequent imaging function, i.e., even if their echo signals are not used for ultrasound imaging, since they are disposed at the edge of the contour 311 rather than at positions close to the middle.

In some embodiments, the contour 311 of the transducer 300 is a polygonal structure, and at least some of the plurality of basic elements 301-304 are disposed at a plurality of vertices of the polygonal structure. A detailed description is provided with reference to FIG. 3. The contour 311 of the transducer 300 may be a quadrilateral structure, and accordingly, the quadrilateral structure has four vertices. The plurality of basic elements 301-304 may be disposed at the four vertices of the polygonal structure, respectively. In such a configuration, only a small number of basic elements at the vertices of the polygon are used to determine the state of acoustic coupling, so that the acoustic coupling condition of the entire transducer 300 can be accurately represented, which saves the consumption of electrical energy and does not affect the imaging function.

Further, it has been described above that whether to activate the imaging function of the transducer is determined according to the reception of the echo signals of two or more of the plurality of basic elements. A more detailed description is provided in conjunction with FIG. 3. As described above, in some embodiments, the imaging function may be activated in response to the echo signals of the ultrasonic signals transmitted by two or more of the plurality of basic elements being detected, and the two or more basic elements include two basic elements that are farthest from each other in the plurality of basic elements. With FIG. 3 as an example, the two basic elements that are farthest from each other may be the basic-element 301 and the basic-element 303. The basic-element 301 and the basic-element 303 are respectively provided at the diagonal vertices of the polygonal contour 311. Such an arrangement can reduce the probability of erroneous determination to a greater extent, since the two basic elements 301 and 303 at the diagonal vertices can represent the acoustic coupling condition of the entire transducer as much as possible, and erroneous determination caused by acoustic coupling generated by only a small part of the transducer is avoided.

It is also described in the above embodiment that the plurality of basic elements for determining whether to activate the imaging function are different from the basic-element for imaging. Taking FIG. 3 as an example, the plurality of basic elements 301-304 are used for assisting in controlling the activation of the imaging function of the transducer 300 as described in any of the embodiments above, and a plurality of imaging basic elements 305 different from the basic elements 301-304 are used for imaging. Such a separate arrangement manner has a number of advantages. For example, a small number of basic elements are used for assisting in controlling the activation of the imaging function, and when the imaging function is not activated, only a small number of basic elements are in operation, so that the electrical energy can be saved and aging of the transducer can be avoided, as much as possible. Further, the functions of the basic elements 301-304 and the imaging basic-element 305 are separate and are not affected by each other, so that the continuity of assisted determination as to whether to activate the imaging function can be ensured, and the continuity of the ultrasound imaging itself can be ensured.

It should be understood that FIG. 3 illustrates the case of a quadrilateral contour 311. However, the shape of the transducer 300 may be other in the art. In addition, the number and position of the basic elements may also be adjusted to some extent under the teachings of the present disclosure. For example, processing the arrangement of the basic elements for assisting in determining at the vertices of the polygon, they may also be arranged at the edges of the polygon. Alternatively, the basic elements may be arranged at some rather than all of the vertices.

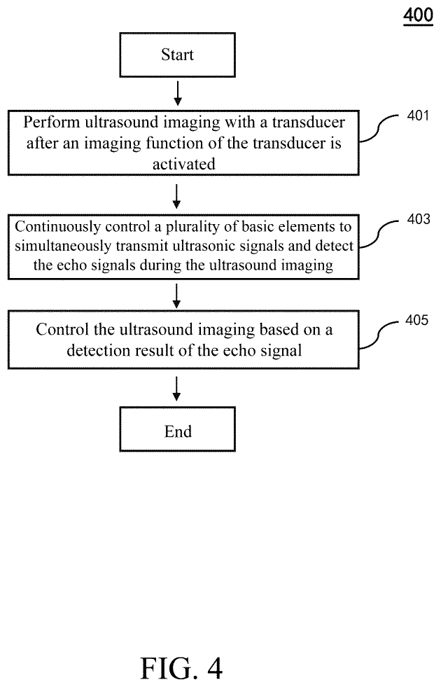

In addition to being able to determine whether to activate the imaging function of the transducer, further provided in some embodiments of the present application is a method for controlling. an imaging function in an imaging process. With reference to FIG. 4, a method 400 for controlling a transducer of an ultrasound probe according to some other embodiments of the present application is shown.

In step 401, ultrasound imaging is performed with the transducer after an imaging function of the transducer is activated, where the manner of activating the imaging function may be as described in any embodiment of the present application, which will not be repeated. When the ultrasound imaging is performed with the transducer, basic elements for imaging may be independent of basic elements for activating the imaging function, such that the imaging function is not affected by the activation function.

In step 403, the plurality of basic elements are continuously controlled to simultaneously transmit ultrasonic signals and detect the echo signals during the ultrasound imaging. As described above, according to the embodiments of the present application, the imaging function and the detection function can be performed simultaneously, so as to provide timely auxiliary information for activation state switching of the transducer of the ultrasound probe.

In step 405, the ultrasound imaging is controlled based on the detection result of the echo signals. In such a configuration, the detection result of the echo signals can be used not only to assist in determining whether to deactivate the imaging function of the transducer, but also to provide timely and effective guidance in the imaging process, further saving the user's workflow. For example, in the process of imaging by a user with the ultrasound probe, it is typically the case that changing a scanned site typically requires moving the ultrasound probe from one position to another over the skin surface, and ending the scanning typically requires moving the ultrasound probe away from the skin surface. In the embodiments of the present application, the use of the ultrasound probe can be monitored in real time so as to control the progress and stop of the ultrasound imaging in time.

How to control the ultrasound imaging based on the detection result of the echo signals is exemplarily described below.

In some embodiments, the ultrasound imaging is continued and alarm information is generated in response to a decrease in the number of basic elements for which echo signals can be detected in the plurality of basic elements, where the arrangement of the plurality of basic elements has been described in detail above. Typically, the decrease in the number of basic elements for which echo signals can be detected means a decrease in the acoustic coupling effect. The inventors have realized that this may occur because the user inadvertently moves the probe that causes the transducer to be in insufficiently close contact with the surface to be scanned. At this time, if the ultrasound imaging is stopped, the scanning efficiency may be lowered. In the embodiments of the present application, when this occurs, on the one hand, the ultrasound imaging is continued to ensure the scanning efficiency, and on the other hand, alarm information is generated to prompt the user to check the coupling state of the ultrasound probe. The alarm information may be in various manners, e.g., a sound signal, a vibration signal, a screen displayed on a display, or the like.

In an alternative embodiment, the ultrasound imaging is stopped in response to an absence of basic elements for which echo signals can be detected in the plurality of basic elements. The inventors have realized that when echo signals can be detected for none of the plurality of basic elements, it typically means a detachment of the transducer from the surface to be scanned, and the user may have completed one scan or may be reselecting a site to be scanned. At this time, controlling the ultrasound imaging to stop can save electrical energy and reduce the degree of aging of the basic elements of the transducer.

It should be understood that the above embodiments may be arbitrarily selected, or may be freely combined and used simultaneously.

The embodiments of the present application provide effective help to control the state of the transducer and save the workflow of the user, and in order to facilitate a better understanding of the present application, some typical usage scenarios are exemplarily described below.

In some usage scenarios, embodiments of the present application are applicable to an ultrasound imaging system to which one wired probe is connected. By means of the solution described in the present application, the user does not need to select activation of the probe in the ultrasound imaging system when performing ultrasound scanning with the one wired probe. When the probe fits to a surface to be scanned, by means of the method according to any of the above embodiments of the present application, it can be quickly determined whether to activate the imaging function and/or the state of the ultrasound probe can be automatically adjusted during and at the end of the imaging process. The method according to the embodiments of the present application further ensures the accuracy of the determination and adjustment described above.

In other usage scenarios, the embodiments of the present application are also applicable to an ultrasound imaging system to which a plurality of wired/wireless probes are connected. By means of the solution described in the present application, the user need not manually select from the plurality of ultrasound probes described above, and the solution of the present application will be able to automatically determine which probe is being effectively used for automatic and accurate activation.

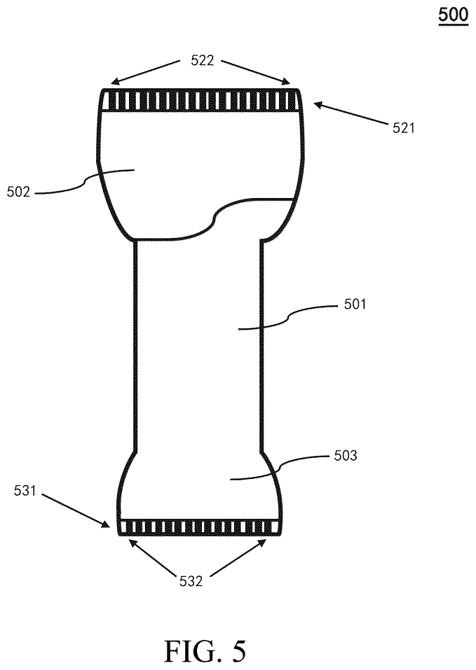

Furthermore, the inventors have also realized that the embodiments of the present application are particularly applicable to automatic activation of a hand-held wireless ultrasound probe having different transducers at two ends. A schematic description is provided below with reference to FIG. 5. FIG. 5 shows a schematic diagram of a wireless ultrasound probe 500 according to some embodiments of the present application.

The probes described above in the present application may include the wireless ultrasound probe 500 shown in FIG. 5. The wireless ultrasound probe 500 includes a grip portion 501. The grip portion 501 includes a first end 502 and a second end 503 that are arranged opposite to each other. The first end 502 and the second end 503 are provided with the transducers 521, 531 and the plurality of basic elements 522, 532, respectively.

The inventors have realized that the wireless ultrasound probe 500 shown in FIG. 5 is particularly suitable for the method described in the embodiments of the present application. The inventors have found that when using the wireless ultrasound probe 500, the user typically needs to determine whether to perform ultrasound scanning with the transducer 521 at the first end 502 or the transducer 531 at the second end 503. Then, the user holds the grip portion 501 with the palm of the hand, and directs one end to be used for scanning downward and the other end upward. For the purpose of holding the probe firmly, the user typically presses the upward end of the probe with the thumb. The pressing of the thumb will cause basic elements of the transducer that are not used to also receive echo signals. In this case, if it is determined which transducer needs to be adopted for imaging only based on the presence or absence of the echo signals, it is inevitable that an erroneous determination is made. In the embodiments of the present application, by detecting and analyzing the echo signal of each of the plurality of basic elements arranged at intervals, the situation described above can be effectively excluded. Since it is difficult for the pressing of the thumb to cover each of the dispersed basic elements, it would be excluded by the control method of the present application. In this way, the degree of accuracy of transducer control at both ends of the wireless ultrasound probe 500 is improved.

In some embodiments, the transducer 521 at the first end 502 and the transducer 531 at the second end 503 are each selected from at least one of a convex array transducer, a linear array transducer, and a phased array transducer. For example, the transducer 521 or transducer 531 may be any one selected from the transducers described above. Alternatively, it is also possible to have a combination of two or more of the transducers described above, and the two or more transducers may be integrated in one transducer at the same end for performing scanning more abundant in functions. In some embodiments, the type of the transducer 521 is different from that of the transducer 531. In such a configuration, the same probe can be applicable to different types of ultrasound scanning, and the user can make more flexible choices according to needs.

Further provided in the present application is an ultrasound imaging system, which may be the ultrasound imaging system as shown in FIG. 1, or may be any other one. The ultrasound imaging system may include: an ultrasound probe and a processor, where the processor is configured to perform the method as described in any embodiment of the present application. The ultrasound probe may also be as described in any embodiment of the present application.

Some embodiments of the present invention further provide a non-transitory computer-readable medium storing a computer program, wherein the computer program has at least one code segment, and the at least one code segment is executable by a machine so that the machine performs steps of the method in any of the embodiments described above.

Correspondingly, the present disclosure may be implemented by means of hardware, software, or a combination of hardware and software. The present disclosure may be implemented in at least one computer system in a centralized manner, or implemented in a distributed manner; and in the distributed manner, different elements are distributed on a plurality of interconnected computer systems. Any type of computer system or other apparatus suitable for implementing the methods described herein is considered to be appropriate.

Various embodiments may also be embedded in a computer program product, which includes all features capable of implementing the methods described herein, and the computer program product is capable of executing these methods when loaded into a computer system. The computer program in this context means any expression in any language, code, or symbol of an instruction set intended to enable a system having information processing capabilities to execute a specific function directly or after any or both of the following: a) conversion to another language, code, or symbol; and b) replication in different material forms.

The purpose of providing the above specific embodiments is to facilitate understanding of the content disclosed in the present invention more thoroughly and comprehensively, but the present invention is not limited to these specific embodiments. Those skilled in the art should understand that various modifications, equivalent replacements, changes and the like can also be made to the present invention and should be included in the scope of protection of the present invention as long as these changes do not depart from the spirit of the present invention.

1. A method for controlling a transducer of an ultrasound probe, comprising:
controlling a plurality of basic elements of the transducer to simultaneously transmit ultrasonic signals, wherein the plurality of basic elements are arranged at intervals;
detecting an echo signal of the ultrasonic signal transmitted by each of the plurality of basic elements; and
controlling an activation state of an imaging function of the transducer based on a detection result of the echo signals.

2. The method according to claim 1, wherein
the plurality of basic elements are arranged at intervals at an edge of a contour of the transducer.

3. The method according to claim 2, wherein
the contour of the transducer is a polygonal structure, and at least some of the plurality of basic elements are arranged at a plurality of vertices of the polygonal structure.

4. The method according to claim 1, wherein the controlling an activation state of an imaging function of the transducer based on a detection result of the echo signals comprises:
in response to the echo signals of the ultrasonic signals transmitted by two or more of the plurality of basic elements being detected, controlling an imaging basic element of the transducer to transmit and receive an ultrasonic signal so as to activate the imaging function; and/or
in response to the echo signal of the ultrasonic signal transmitted by one or fewer of the plurality of basic elements being detected, controlling an imaging basic element of the transducer not to transmit or receive an ultrasonic signal so as not to activate the imaging function.

5. The method according to claim 4, wherein
the plurality of basic elements are different from the imaging basic element.

6. The method according to claim 4, wherein
the two or more basic elements comprise two basic elements that are farthest from each other in the plurality of basic elements.

7. The method according to claim 1, further comprising:
performing ultrasound imaging with the transducer after the imaging function of the transducer is activated;
continuously controlling the plurality of basic elements to simultaneously transmit ultrasonic signals and detect the echo signals during the ultrasound imaging; and
controlling the ultrasound imaging based on a detection result of the echo signals.

8. The method according to claim 7, wherein the controlling the ultrasound imaging based on a detection result of the echo signals comprises:
in response to a decrease in the number of basic elements for which echo signals can be detected in the plurality of basic elements, continuing to perform ultrasound imaging and generating alarm information; and/or
stopping the ultrasound imaging in response to an absence of basic elements for which echo signals can be detected in the plurality of basic elements.

9. The method according to claim 1, wherein
the ultrasound probe comprises a wireless ultrasound probe, the wireless ultrasound probe comprises a grip portion, the grip portion comprises a first end and a second end that are arranged opposite to each other, and the first end and the second end are each provided with the transducer and the plurality of basic elements.

10. The method according to claim 9, wherein
the transducer at the first end and the transducer at the second end are each selected from at least one of a convex array transducer, a linear array transducer, and a phased array transducer.

11. The method according to claim 10, wherein
the transducer at the first end is different from the transducer at the second end.

12. An ultrasound imaging system, comprising:
an ultrasound probe, comprising a transducer;
a memory storing instructions; and
a processor configured to execute the instructions to:
control a plurality of basic elements of the transducer to simultaneously transmit ultrasonic signals, wherein the plurality of basic elements are arranged at intervals;
detect an echo signal of the ultrasonic signal transmitted by each of the plurality of basic elements; and
control an activation state of an imaging function of the transducer based on a detection result of the echo signals.

13. A non-transitory computer-readable medium, the non-transitory computer-readable medium having a computer program stored therein, the computer program having at least one code segment, and the at least one code segment being executable by a machine to cause the machine to execute the steps of:
controlling a plurality of basic elements of the transducer to simultaneously transmit ultrasonic signals, wherein the plurality of basic elements are arranged at intervals;
detecting an echo signal of the ultrasonic signal transmitted by each of the plurality of basic elements; and
controlling an activation state of an imaging function of the transducer based on a detection result of the echo signals.
